(19) United States
(12) Patent Application Publication
(10) Pub. No.: US 2024/0002365 A1
(43) Pub. Date: Jan. 4, 2024

(54) PYRIDAZINE AND 1,2,4-TRIAZINE DERIVATIVES AS FGFR KINASE INHIBITORS

(71) Applicant: BETA PHARMA, INC., Wilmington, DE (US)

(72) Inventors: Don Zhang, Princeton, NJ (US); Jirong Peng, Mequon, WI (US); Michael John Costanzo, Bonney Lake, WA (US); Michael Alan Green, Easton, PA (US); Michael Nicholas Greco, Lansdale, PA (US)

(21) Appl. No.: 17/793,302

(22) PCT Filed: Jan. 12, 2021
(86) PCT No.: PCT/US2021/013038

§ 371 Date: Jul. 15, 2022

Related U.S. Application Data
(60) Provisional application No. 62/962,396, filed on Jan. 17, 2020.

(51) Int. Cl. C07D 403/04

(52) U.S. Cl. CPC C07D 403/04

(57) ABSTRACT

The present invention is directed to inhibitors of fibroblast growth factors, and more particularly to compounds of Formula (I), as well as compositions comprising Formula (I) and methods of using the compound of Formula (I) for the treatment or prevention of a disease, disorder, or medical condition mediated through the fibroblast growth factor receptor (FGFR), especially FGFR1-4. These diseases, disorders, or medical conditions include various cancers.

### CROSS-REFERENCE TO RELATED APPLICATIONS

This application claims the benefit of U.S. Provisional Application Ser. No. 62/962,396 filed Jan. 17, 2020, which is incorporated by reference herein in its entirety.

### FIELD OF THE INVENTION

The present invention is directed to inhibitors of fibroblast growth factors, and more particularly to compounds, compositions and methods for the treatment or prevention of a disease, disorder, or medical condition mediated through the fibroblast growth factor receptor (FGFR), especially FGFR1-4. These diseases, disorders, or medical conditions include various cancers.

### BRIEF DESCRIPTION OF THE RELATED ART

Fibroblast growth factors (FGFs) are a family of cell-signaling proteins that are mediators of numerous physiological processes required for normal development. There are at least 22 known FGFs that control processes in both developmental and mature tissue maintenance. FGFs are controlled by fibroblast growth factor receptors (FGFRs). The fibroblast growth factor receptors are a family of receptor tyrosine kinases containing four members; FGFR1, FGFR2, FGFR3 and FGFR4, each of which shares a high degree of sequence homology. FGFRs are activated by FGFs that control cell proliferation, migration, apoptosis and differentiation.

Various tumor types have been found to contain genetic alterations in FGF and FGFR leading to dysregulation in signaling and ultimately cancer. Overexpression and abnormalities in the FGF/FGFR families have been associated with bladder, renal, gastric, squamous cell lung cancer and multiple myeloma. Protein overexpression and gene amplification in FGFR1, FGFR2 and FGFR4 in breast cancer has been reported, thus underscoring the issue of receptor selectivity (J. Med. Chem. 2011, 54, 7066). As a consequence of the direct association between FGF signaling and numerous cancers, compounds that target FGF signaling and FGFRs represent potential agents for the treatment of various cancers. Several small molecule FGFR inhibitors have reached human clinical trials, however there remains a need for selective small molecule FGFR inhibitors.

### SUMMARY OF THE INVENTION

In one aspect, the present invention is directed to a compound of Formula I:

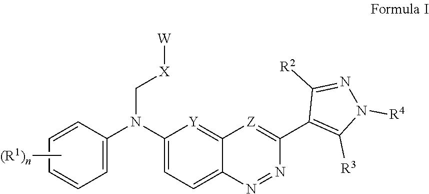

or a pharmaceutically acceptable salt, solvate, or prodrug thereof, wherein:
W is selected from H, OH, NH₂, NH(C₁₋₆alkyl), 1-azetindinyl, 1-pyrrolidinyl, 1-piperidinyl, 2-pyridyl, 1,2,3,6-tetrahydro-1-pyridinyl, 2-pyrimidinyl, 4-pyrimidinyl, 2-thiazolo, and phenyl optionally substituted with one or more of OH, halogen, CF₃, C₁₋₄alkyl, O(C₁₋₄alkyl), (C₁₋₄alkyl)OH, CN, CH₂CN, C(O)NH₂, C(O)NH(C₁₋₄alkyl), C(O)N(C₁₋₄alkyl)₂, NH₂, NH(C₁₋₄alkyl), N(C₁₋₄alkyl)₂, NHSO₂(C₁₋₄alkyl), NHSO₂NH(C₁₋₄alkyl) and NHSO₂N(C₁₋₄alkyl)₂;
X is selected from a bond, methylene, ethylene, and ethynylene;
Y and Z are the same or different and are selected from CH and N;
each R¹ is independently selected from is independently selected from H, OH, halogen, CN, C₁₋₄alkyl, O(C₁₋₄alkyl), NH(C₁₋₄alkyl), and N(C₁₋₄alkyl)₂;
n is an integer selected from 1-5;
R² and R³ are the same or different and are selected from H and C₁₋₄alkyl; and
R⁴ is selected from H, C₁₋₄alkyl, C₃₋₆cycloalkyl, C₁₋₄alkylOH, CH₂C(O)NHC₁₋₂alkyl, CH₂C(O)N(C₁₋₂alkyl)₂, CH₂(SO)₂C₁₋₂alkyl, 4-(λ³-methyl)tetrahydro-2H-pyran, 4-(λ³-methyl)piperidine, CH₂P(O)(C₁₋₂alkyl)₂, CH₂P(O)(OC₁₋₂alkyl)₂, CH₂P(O)(OC₁₋₂alkyl)OH and CH₂P(O)(OH)₂.

In another aspect, the present invention is directed to a compound of Formula II:

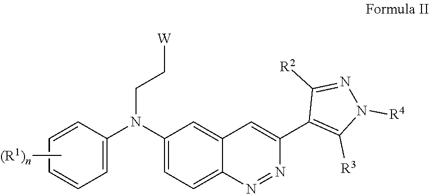

or a pharmaceutically acceptable salt, solvate, or prodrug thereof, wherein:
W is selected from H, OH, NH₂, NH(C₁₋₆alkyl), 1-azetindinyl, 1-pyrrolidinyl, 1-piperidinyl, 2-pyridyl, 1,2,3,6-tetrahydro-1-pyridinyl, 2-pyrimidinyl, 4-pyrimidinyl, 2-thiazolo, and phenyl optionally substituted with one or more of OH, halogen, CF₃, C₁₋₄alkyl, O(C₁₋₄alkyl), (C₁₋₄alkyl)OH, CN, CH₂CN, C(O)NH₂, C(O)NH(C₁₋₄alkyl), C(O)N(C₁₋₄alkyl)₂, NH₂, NH(C₁₋₄alkyl), N(C₁₋₄alkyl)₂, NHSO₂(C₁₋₄alkyl), NHSO₂NH(C₁₋₄alkyl) and NHSO₂N(C₁₋₄alkyl)₂;
each R¹ is independently selected from is independently selected from H, OH, halogen, CN, C₁₋₄alkyl, O(C₁₋₄alkyl), NH(C₁₋₄alkyl), and N(C₁₋₄alkyl)₂;
n is an integer selected from 1-5;
R² and R³ are the same or different and are selected from H and C₁₋₄alkyl; and
R⁴ is selected from H, C₁₋₄alkyl, C₁₋₄alkylOH, C₃₋₆cycloalkyl, CH₂C(O)NHC₁₋₂alkyl, CH₂C(O)N(C₁₋₂alkyl)₂, CH₂(SO)₂C₁₋₂alkyl, 4-(λ³-methyl)tetrahydro-2H-pyran, 4-(λ³-methyl)piperidine, CH₂P(O)(C₁₋₂alkyl)₂, CH₂P(O)(OC₁₋₂alkyl)₂, CH₂P(O)(OC₁₋₂alkyl)OH and CH₂P(O)(OH)₂.

In another aspect, the present invention is directed to a compound of Formula III:

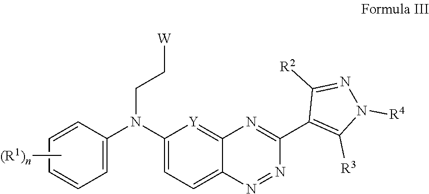

or a pharmaceutically acceptable salt, solvate, or prodrug thereof, wherein:
W is selected from H, OH, NH₂, NH(C₁₋₆alkyl), 1-azetindinyl, 1-pyrrolidinyl, 1-piperidinyl, 2-pyridyl, 1,2,3,6-tetrahydro-1-pyridinyl, 2-pyrimidinyl, 4-pyrimidinyl, 2-thiazolo, and phenyl optionally substituted with one or more of OH, halogen, CF₃, C₁₋₄alkyl, O(C₁₋₄alkyl), (C₁₋₄alkyl)OH, CN, CH₂CN, C(O)NH₂, C(O)NH(C₁₋₄alkyl), C(O)N(C₁₋₄alkyl)₂, NH₂, NH(C₁₋₄alkyl), N(C₁₋₄alkyl)₂, NHSO₂(C₁₋₄alkyl), NHSO₂NH(C₁₋₄alkyl) and NHSO₂N(C₁₋₄alkyl)₂;
X is selected from a bond, methylene, ethylene, and ethynylene;
Y is selected from CH and N;
each R¹ is independently selected from is independently selected from H, OH, halogen, CN, C₁₋₄alkyl, O(C₁₋₄alkyl), NH(C₁₋₄alkyl), and N(C₁₋₄alkyl)₂;
n is an integer selected from 1-5;
R² and R³ are the same or different and are selected from H and C₁₋₄alkyl; and
R⁴ is selected from H, C₁₋₄alkyl, C₃₋₆cycloalkyl, C₁₋₄alkylOH, CH₂C(O)NHC₁₋₂alkyl, CH₂C(O)N(C₁₋₂alkyl)₂, CH₂(SO)₂C₁₋₂alkyl, 4-(λ³-methyl)tetrahydro-2H-pyran, 4-(λ³-methyl)piperidine, CH₂P(O)(C₁₋₂alkyl)₂, CH₂P(O)(OC₁₋₂alkyl)₂, CH₂P(O)(OC₁₋₂alkyl)OH and CH₂P(O)(OH)₂.

In another aspect, the present invention is directed to a pharmaceutical composition comprising a compound or salt of Formulae I, II, or III together with a pharmaceutically acceptable carrier.

In another aspect, the present invention is directed to a method of treating a disease, disorder, or medical condition in a patient, comprising the step of providing to a patient in need thereof a therapeutic agent, wherein the therapeutic agent comprises the compound of Formulae I, II, or III, or salt thereof.

These and other aspects come apparent upon reading the following detailed description of the invention.

### BRIEF DESCRIPTION OF THE DRAWINGS

FIG. 1 illustrates a time course of Plasma Concentration of Example Compound 1 after IV Dosing at 1 mg/kg; and

FIG. 2 illustrates a time course of Plasma Concentration of Example Compound 1 after IV Dosing at 10 mg/kg.

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

All Formulae disclosed herein include all pharmaceutically acceptable salts of such Formulae.

The opened ended term “comprising” includes the intermediate and closed terms “consisting essentially of” and “consisting of.”

The term “substituted” means that any one or more hydrogens on the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent.

“Alkyl” includes both branched, cyclo-, and straight chain saturated aliphatic hydrocarbon groups, having the specified number of carbon atoms, generally from 1 to about 8 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g. C₁-C₈alkyl, C₁-C₄alkyl, and C₁-C₂alkyl. When C₀-Cₙ alkyl is used herein in conjunction with another group, for example, —C₀-C₂alkyl(phenyl), the indicated group, in this case phenyl, is either directly bound by a single covalent bond (C₀alkyl), or attached by an alkyl chain having the specified number of carbon atoms, in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, 3-methylbutyl, t-butyl, n-pentyl, and sec-pentyl.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “Alkylthio” or a “thioalkyl” group is an alkyl group as defined above with the indicated number of carbon atoms covalently bound to the group it substitutes by a sulfur bridge (—S—). Similarly, “alkenyloxy”, “alkynyloxy”, and “cycloalkyloxy” refer to alkenyl, alkynyl, and cycloalkyl groups, in each instance covalently bound to the group it substitutes by an oxygen bridge (—O—).

“Halo” or “halogen” means fluoro, chloro, bromo, or iodo, and are defined herein to include all isotopes of same, including heavy isotopes and radioactive isotopes. Examples of useful halo isotopes include ¹⁸F, ⁷⁶Br, and ¹³¹I. Additional isotopes are readily appreciated by one of skill in the art.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

“Peptide” means a molecule which is a chain of amino acids linked together via amide bonds (also called peptide bonds).

“Pharmaceutical compositions” means compositions comprising at least one active agent, such as a compound or salt of Formula II, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. In certain embodiments treatment of the disease may be commenced before the patient presents symptoms of the disease.

A “therapeutically effective amount” of a pharmaceutical composition means an amount effective, when administered to a patient, to provide a therapeutic benefit such as an amelioration of symptoms, decrease disease progression, or cause disease regression.

A “therapeutic compound” means a compound which can be used for diagnosis or treatment of a disease. The compounds can be small molecules, peptides, proteins, or other kinds of molecules.

A significant change is any detectable change that is statistically significant in a standard parametric test of statistical significance such as Student's T-test, where p<0.05.

### Chemical Description

Compounds of the Formulae disclosed herein may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts) of an active agent may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers may exist as atropisomers. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

A “chelating group” or “chelator” is a ligand group which can form two or more separate coordinate bonds to a single central atom, which is usually a metal ion. Chelating groups as disclosed herein are organic groups which possess multiple N, O, or S heteroatoms, and have a structure which allows two or more of the heteroatoms to form bonds to the same metal ion.

“Pharmaceutically acceptable salts” include derivatives of the disclosed compounds in which the parent compound is modified by making inorganic and organic, non toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

The compounds of the present invention relate to certain pyridazine and 1,2,4-triazine derivatives of Formula I, II, or III, or pharmaceutically acceptable salts, solvates, or prodrugs thereof. The compounds of this invention are useful in the treatment or prevention of diseases, disorders, or medical conditions mediated through certain FGFR signaling pathways, such as various cancers.

One aspect of the present invention is directed to pyridazine and 1,2,4-triazine derivatives of Formula I, or pharmaceutically acceptable salts, solvates, or prodrugs thereof, as inhibitors of fibroblast growth factor receptors:

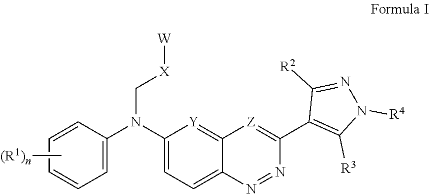

In Formula I, W is selected from H, OH, NH₂, NH(C₁₋₆alkyl), 1-azetindinyl, 1-pyrrolidinyl, 1-piperidinyl, 2-pyridyl, 1,2,3,6-tetrahydro-1-pyridinyl, 2-pyrimidinyl, 4-pyrimidinyl, 2-thiazolo, and phenyl optionally substituted with one or more of OH, halogen, CF₃, C₁₋₄alkyl, O(C₁₋₄alkyl), (C₁₋₄alkyl)OH, CN, CH₂CN, C(O)NH₂, C(O)NH(C₁₋₄alkyl), C(O)N(C₁₋₄alkyl)₂, NH₂, NH(C₁₋₄alkyl), N(C₁₋₄alkyl)₂, NHSO₂(C₁₋₄alkyl), NHSO₂NH(C₁₋₄alkyl) and NHSO₂N(C₁₋₄alkyl)₂; X is selected from a bond, methylene, ethylene, and ethynylene; Y and Z are the same or different and are selected from CH and N; each R¹ is independently selected from is independently selected from H, OH, halogen, CN, C₁₋₄alkyl, O(C₁₋₄alkyl), NH(C₁₋₄alkyl), and N(C₁₋₄alkyl)₂; n is an integer selected from 1-5; R² and R³ are the same or different and are selected from H and C₁₋₄alkyl; and R⁴ is selected from H, C₁₋₄alkyl, C₃₋₆cycloalkyl, C₁₋₄alkkylOH, CH₂C(O)NHC₁₋₂alkyl, CH₂C(O)N(C₁₋₂alkyl)₂, CH₂(SO)₂C₁₋₂alkyl, 4-(λ³-methyl)tetrahydro-2H-pyran, 4-(λ³-methyl)piperidine, CH₂P(O)(C₁₋₂alkyl)₂, CH₂P(O)(OC₁₋₂alkyl)₂, CH₂P(O)(OC₁₋₂alkyl)OH and CH₂P(O)(OH)₂.

One preferred embodiment of the compounds of the invention shown in Formula I is the compound illustrated in Formula II:

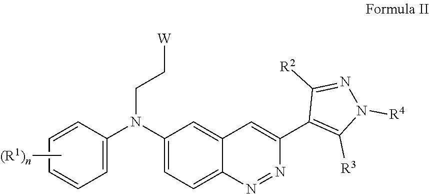

or a pharmaceutically acceptable salt, solvate, or prodrug thereof.

In Formula II, W is selected from H, OH, NH₂, NH(C₁₋₆alkyl), 1-azetindinyl, 1-pyrrolidinyl, 1-piperidinyl, 2-pyridyl, 1,2,3,6-tetrahydro-1-pyridinyl, 2-pyrimidinyl, 4-pyrimidinyl, 2-thiazolo, and phenyl optionally substituted with one or more of OH, halogen, CF₃, C₁₋₄alkyl, O(C₁₋₄alkyl), (C₁₋₄alkyl)OH, CN, CH₂CN, C(O)NH₂, C(O)NH(C₁₋₄alkyl), C(O)N(C₁₋₄alkyl)₂, NH₂, NH(C₁₋₄alkyl), N(C₁₋₄alkyl)₂, NHSO₂(C₁₋₄alkyl), NHSO₂NH(C₁₋₄alkyl) and NHSO₂N(C₁₋₄alkyl)₂; each R¹ is independently selected from is independently selected from H, OH, halogen, CN, C₁₋₄alkyl, O(C₁₋₄alkyl), NH(C₁₋₄alkyl), and N(C₁₋₄alkyl)₂; n is an integer selected from 1-5; R² and R³ are the same or different and are selected from H and C₁₋₄alkyl; and R⁴ is selected from H, C₁₋₄alkyl, C₃₋₆cycloalkyl, C₁₋₄alkylOH, CH₂C(O)NHC₁₋₂alkyl, CH₂C(O)N(C₁₋₂alkyl)₂, CH₂(SO)₂C₁₋₂alkyl, 4-(λ³-methyl)tetrahydro-2H-pyran, 4-(λ³-methyl)piperidine, CH₂P(O)(C₁₋₂alkyl)₂, CH₂P(O)(OC₁₋₂alkyl)₂, CH₂P(O)(OC₁₋₂alkyl)OH and CH₂P(O)(OH)₂.

Particularly preferred species of the present invention as illustrated in Formula II include the following:

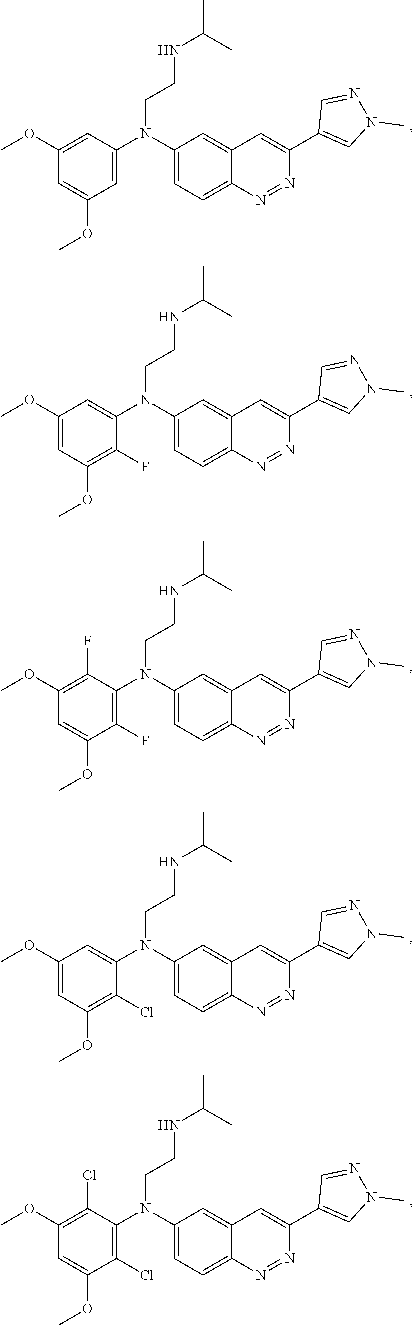

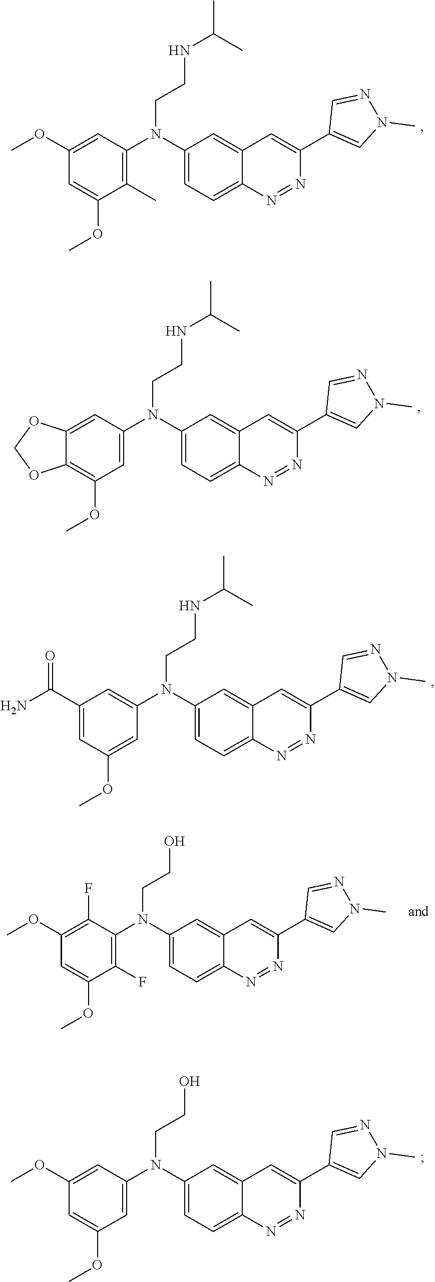

or pharmaceutically acceptable salts, solvates, or prodrugs thereof.

Another preferred embodiment of the compounds of the invention is shown in Formula III:

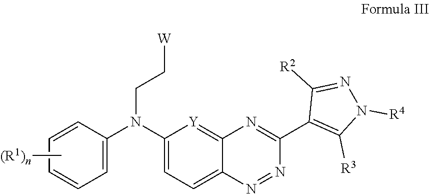

or a pharmaceutically acceptable salt, solvate, or prodrug thereof.

In Formula III, W is selected from H, OH, NH₂, NH(C₁₋₆alkyl), 1-azetindinyl, 1-pyrrolidinyl, 1-piperidinyl, 2-pyridyl, 1,2,3,6-tetrahydro-1-pyridinyl, 2-pyrimidinyl, 4-pyrimidinyl, 2-thiazolo, and phenyl optionally substituted with one or more of OH, halogen, CF₃, C₁₋₄alkyl, O(C₁₋₄alkyl), (C₁₋₄alkyl)OH, CN, CH₂CN, C(O)NH₂, C(O)NH(C₁₋₄alkyl), C(O)N(C₁₋₄alkyl)₂, NH₂, NH(C₁₋₄alkyl), N(C₁₋₄alkyl)₂, NHSO₂(C₁₋₄alkyl), NHSO₂NH(C₁₋₄alkyl) and NHSO₂N(C₁₋₄alkyl)₂; X is selected from a bond, methylene, ethylene, and ethynylene; Y is selected from CH and N; each R¹ is independently selected from is independently selected from H, OH, halogen, CN, C₁₋₄alkyl, O(C₁₋₄alkyl), NH(C₁₋₄alkyl), and N(C₁₋₄alkyl)₂; n is an integer selected from 1-5; R² and R³ are the same or different and are selected from H and C₁₋₄alkyl; and R⁴ is selected from H, C₁₋₄alkyl, C₃₋₆cycloalkyl, C₁₋₄alkylOH, CH₂C(O)NHC₁₋₂alkyl, CH₂C(O)N(C₁₋₂alkyl)₂, CH₂(SO)₂C₁₋₂alkyl, 4-(λ³-methyl)tetrahydro-2H-pyran, 4-(λ³-methyl)piperidine, CH₂P(O)(C₁₋₂alkyl)₂, CH₂P(O)(OC₁₋₂alkyl)₂, CH₂P(O)(OC₁₋₂alkyl)OH and CH₂P(O)(OH)₂.

Particularly preferred species of the present invention as illustrated in Formula II include the following:

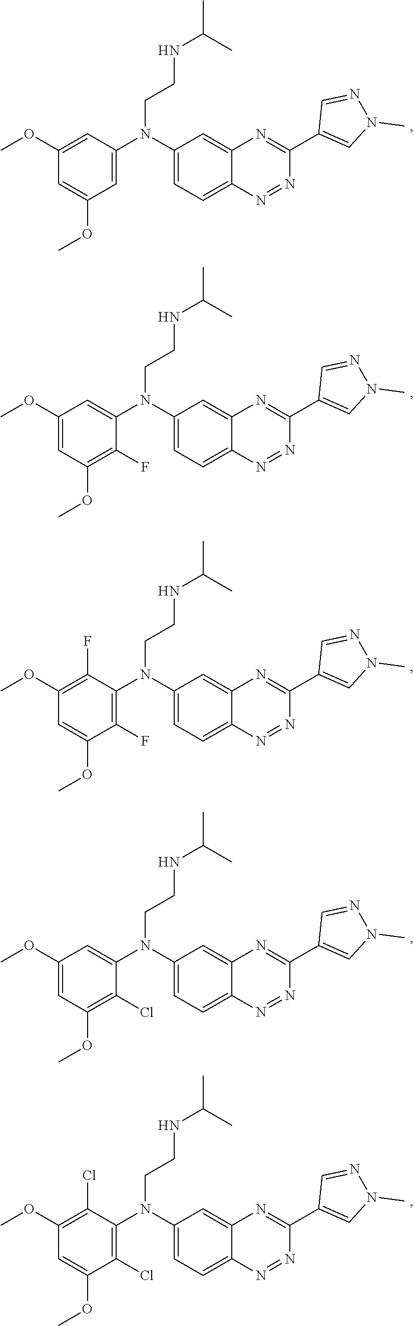

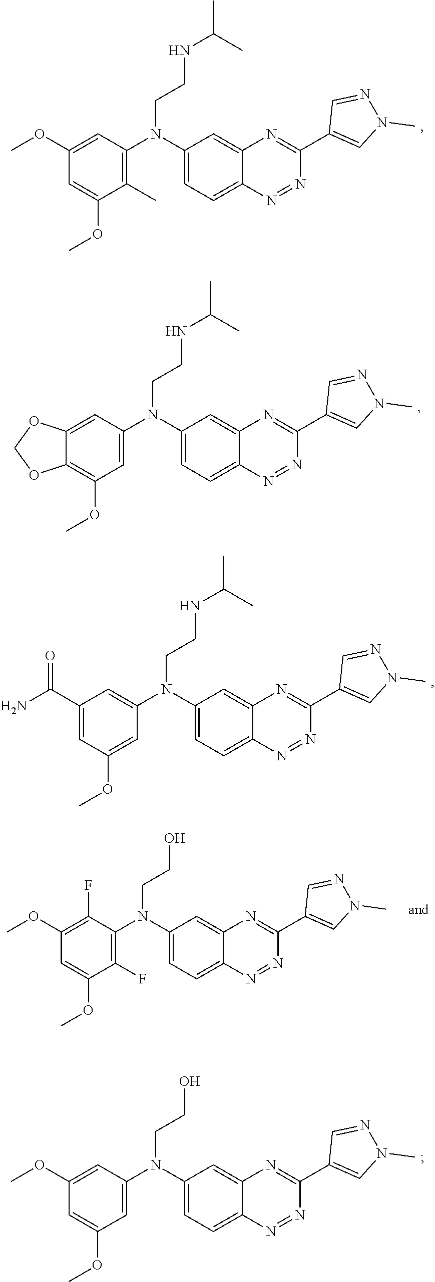

or pharmaceutically acceptable salts, solvates, or prodrugs thereof.

Additional preferred species of the compound of Formula III include the following:

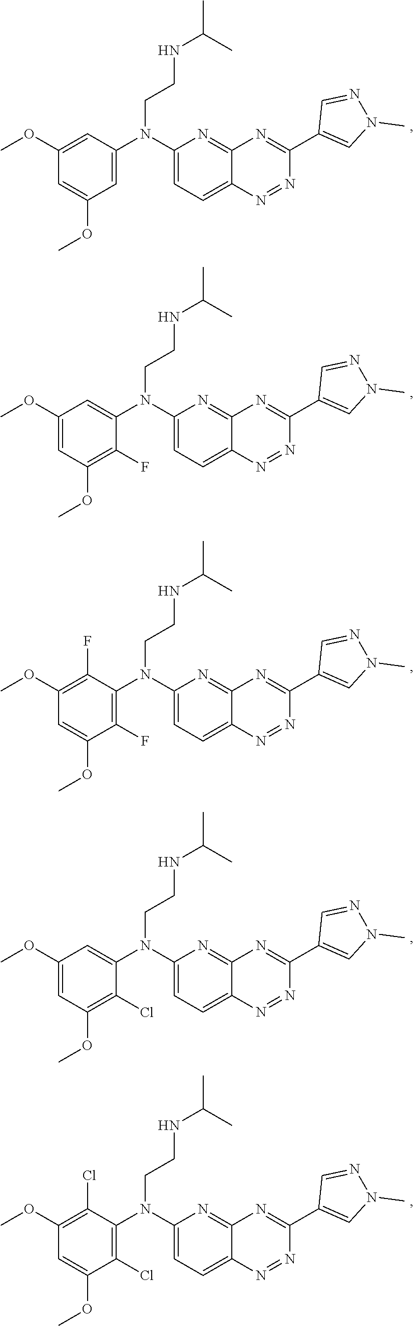

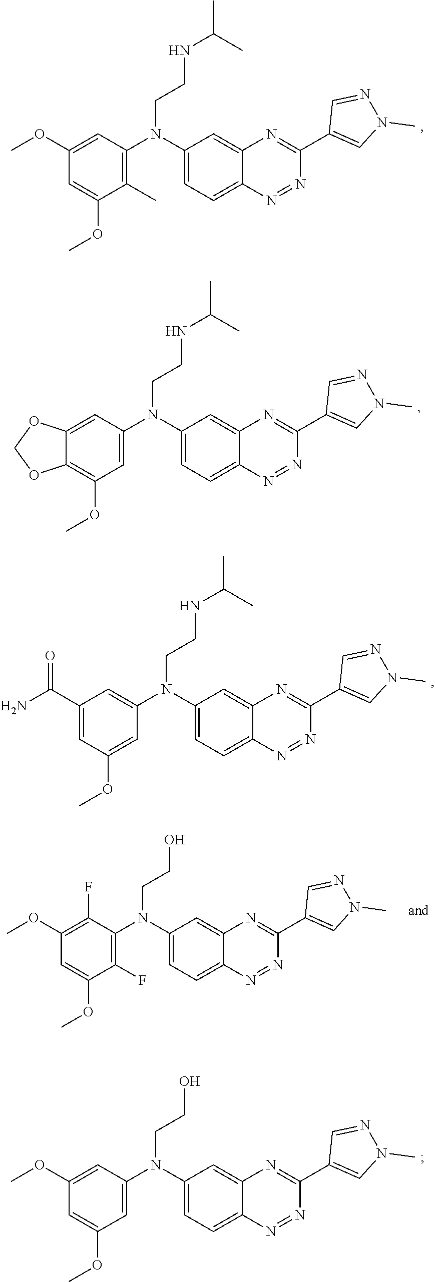

or pharmaceutically acceptable salts, solvates, or prodrugs thereof.

In yet another aspect, the present invention is directed to a pharmaceutical composition comprising a compound, salt, solvate, or prodrug of any one of Formulae I, II, or III, together with a pharmaceutically acceptable carrier.

In yet another aspect, the present invention is directed to a method of treating a disease, disorder, or medical condition in a patient, comprising the step of providing or administering to a patient in need thereof a therapeutic agent, wherein the therapeutic agent is a compound, salt, solvate, or prodrug thereof of any one of Formulae I, II, or III. The method of treating a disease, disorder, or medical condition includes treating a patient suffering from various cancers, including, but not limited to from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer, pancreatic cancer, and the like.

The method of treating a disease, disorder, or medical condition also includes treating a patient suffering from diseases, disorders, or medical conditions that are mediated through the fibroblast growth factor receptor (FGFR), and particularly through FGFR1-4.

Compounds disclosed herein can be administered as the neat chemical, but are preferably administered as a pharmaceutical composition. Accordingly, the invention encompasses pharmaceutical compositions comprising a compound or pharmaceutically acceptable salt of a compound, such as a compound of Formulae I, II, or III, together with at least one pharmaceutically acceptable carrier. The pharmaceutical composition may contain a compound or salt of Formulae I, II, or III as the only active agent, but is preferably contains at least one additional active agent. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of Formulae I, II, or III and optionally from about 0.1 mg to about 2000 mg, from about 10 mg to about 1000 mg, from about 100 mg to about 800 mg, or from about 200 mg to about 600 mg of an additional active agent in a unit dosage form. The pharmaceutical composition may also include a molar ratio of a compound, such as a compound of Formulae I, II, or III and an additional active agent. For example the pharmaceutical composition may contain a molar ratio of about 0.5:1, about 1:1, about 2:1, about 3:1 or from about 1.5:1 to about 4:1 of an additional active agent to a compound of Formulae I, II, or III.

Compounds disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the compound of the present invention.

The pharmaceutical compositions/combinations can be formulated for oral administration. These compositions contain between 0.1 and 99 weight % (wt. %) of a compound of Formulae I, II, or III. Some embodiments contain from about 25 wt % to about 50 wt % or from about 5 wt % to about 75 wt % of the compound of Formulae I, II, or III.

### Treatment Methods

As indicated above, the compounds of Formulae I, II, and III, as well as pharmaceutical compositions comprising the compounds, are useful for diagnosis or treatment of a disease, disorder, or medical condition mediated through FGFR, especially FGFR1-4, and including various cancers, such as glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer or pancreatic cancer.

According to the present invention, a method of FGFR-mediated diseases or conditions comprises providing to a patient in need of such treatment a therapeutically effective amount of a compound of Formula I, II, or III. In one embodiment, the patient is a mammal, and more specifically a human. As were understood by one skilled in the art, the invention also encompasses methods of treating non-human patients such as companion animals, e.g. cats, dogs, and livestock animals.

A therapeutically effective amount of a pharmaceutical composition is preferably an amount sufficient to reduce or ameliorate the symptoms of a disease or condition. In the case of FGFR-mediated diseases for example, a therapeutically effective amount may be an amount sufficient to reduce or ameliorate cancer. A therapeutically effective amount of a compound or pharmaceutical composition described herein will also provide a sufficient concentration of a compound of Formula I, II, or III when administered to a patient. A sufficient concentration is preferably a concentration of the compound in the patient's body necessary to prevent or combat the disorder. Such an amount may be ascertained experimentally, for example by assaying blood concentration of the compound, or theoretically, by calculating bioavailability.

According to the invention, the methods of treatment disclosed herein include providing certain dosage amounts of a compound of Formula I, II, or III to a patient. Dosage levels of each compound of from about 0.01 mg to about 100 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a compound of Formula I, II, or III are provided daily to a patient. Frequency of dosage may also vary depending on the compound used and the particular disease treated. However, for treatment of most FGFR-mediated diseases and disorders, a dosage regimen of 4 times daily or less can be used and in certain embodiments a dosage regimen of 1 or 2 times daily is used.

It were understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

A compound of Formula I, II, or III may be administered singularly (i.e., sole therapeutic agent of a regime) to treat or prevent FGFR-mediated diseases and conditions such as various cancers, or may be administered in combination with another active agent. One or more compounds of Formula I, II, or III may be administered in coordination with a regime of one or more other active agents such as anticancer cytotoxic agents. In an embodiment, a method of treating or diagnosing FGFR-mediated cancer in a mammal includes administering to said mammal a therapeutically effective amount of a compound of Formula I, II, or III, optionally in combination with one or more additional active ingredients.

As were appreciated by one skilled in the art, the methods of treatment provided herein are also useful for treatment of mammals other than humans, including for veterinary applications such as to treat horses and livestock, e.g. cattle, sheep, cows, goats, swine and the like, and pets (companion animals) such as dogs and cats.

For diagnostic or research applications, a wide variety of mammals were suitable subjects including rodents (e.g. mice, rats, hamsters), rabbits, primates, and swine such as inbred pigs and the like. Additionally, for in vitro applications, such as in vitro diagnostic and research applications, body fluids (e.g. blood, plasma, serum, cellular interstitial fluid, saliva, feces, and urine) and cell and tissue samples of the above subjects were suitable for use.

In one embodiment, the invention provides a method of treating a disease, disorder, or medical condition mediated through FGFR, especially FGFR1-4, including various cancers, in a patient identified as in need of such treatment, the method comprising providing to the patient an effective amount of a compound of Formula I, II, or III. The compounds of Formulae I, II, or III provided herein may be administered alone, or in combination with one or more other active agents.

In another embodiment, the method of treating or diagnosing FGFR-mediated diseases or conditions may additionally comprise administering the compound of Formulae I, II, or III in combination with one or more additional compounds, wherein at least one of the additional compounds is an active agent, to a patient in need of such treatment. The one or more additional compounds may include additional therapeutic compounds, including anticancer therapeutic compounds such as doxorubicin, paclitaxel, docetaxel, cisplatin, camptothecin, temozolomide, avastin, Herceptin, Erbitux, and the like.

### EXAMPLES

### Chemical Synthesis

The compounds of the Formulae I, II, or III described herein, and/or the pharmaceutically acceptable salts thereof, can be synthesized from commercially available starting materials by methods well known to those skilled in the art of synthetic organic chemistry. The following general synthetic Schemes 1 and 2 illustrates representative methods to prepare most of the example compounds. The compounds thus obtained can be further modified at their peripheral positions to provide the desired compounds. Synthetic chemistry transformations are described, for example, in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof. The compounds of the Formula I and/or their pharmaceutically acceptable salts described herein can be purified by column chromatography, high performance liquid chromatography, crystallization, or other suitable methods. The listed starting materials, reactions, reagents, solvents, temperatures, catalysts and ligands are not limited to what is depicted for purely illustrative purposes. Certain abbreviations and acronyms well known to those trained in the art that may be used in Schemes 1 and 2 and in the following examples are listed below for clarity.

### Abbreviations and Acronyms

The following abbreviations and acronyms may be used in this application:
anhyd.=anhydrous;
aq.=aqueous;
B₂pin₂=bis(pinacolato)diboron;
Boc=tert-butoxycarbonyl;
n-Bu₃P=tri-n-butylphosphine;
CAS #=Chemical Abstracts Service Registry Number;
Compd=compound;
d=day(s);
DCM=dichloromethane;
DIEA=DIPEA=N,N-diisopropylethylamine;
DME=1,2-dimethoxyethane;
DMF=N,N-dimethylformamide;
DMSO=dimethylsulfoxide;
DMA=N,N-dimethylacetamide;
dppf=1,1′-bis(diphenylphosphino)ferrocene)
EtOAc=ethyl acetate;
Ex=Example;
FCC=flash column chromatography using silica;
h=hour(s);
KHMDS=potassium bis(trimethylsilyl)amide [KN(SiMe₃)₂];
LDA=lithium diisopropylamide;
LiHMDS=lithium bis(trimethylsilyl)amide [LiN(SiMe₃)₂];
MeOH=methanol;
min.=minutes;
Pd₂(dba)₃=tris(dibenzylideneacetone)dipalladium(0);
Pd(dppf)Cl₂=[1,1′-bis(diphenylphosphino)ferrocene]dichloropalladium(II);
RT=room temperature;
RuPhos=2-dicyclohexylphosphino-2′,6′-diisopropoxybiphenyl;
RuPhos Pd Gen 1·t-BuOMe=chloro-(2-dicyclohexylphosphino-2′,6′-diisopropoxy-1,1′-biphenyl)[2-(2-aminoethyl)phenyl]palladium(II)-methyl-t-butyl ether adduct;
satd.=saturated solution;
TEA=triethylamine;
TFA=trifluoroacetic acid;
THE=tetrahydrofuran;
Xantphos=4,5-bis(diphenylphosphino)-9,9-dimethylxanthene (CAS #161265-03-8).

Scheme 1 begins with the diazotization of 1-(2-amino-5-bromophenyl)ethanone (2a) with NaNO₂ in 5 N HCl and cyclization of the intermediate diazonium salt to generate cinnoline 2b. Bromination of 2b with Br₂ and sodium acetate in acetic acid furnishes compound 2c. Chlorination of 2c with a mixture of PCl₅ and POCl₃ provides 3,4-dichlorocinnoline 2d, which reacts at room temperature with hydrazine to give 2e. Reduction of compound 2e with aqueous CuSO₄ in methanol furnishes 6-bromo-3-chlorocinnoline (2f). Buchwald-Hartwig amination of 2f with aniline 2g provides 2h, which is subsequently coupled with boronate ester 2i to give compound 2j. Desilylation of 2j with tetrabutylammonium fluoride yields alcohol 2k. Reaction of 2k with methanesulfonyl chloride in the presence of triethylamine generates the mesylate 2m. Finally, reaction of 2m with W—H produces compounds of the Formula II.

Scheme 2 starts with the reaction of fluoro compound 3a with guanidinium chloride in the presence of potassium tert-butoxide to furnish 3b. Diazotization of 3b in aqueous H₂SO₄ forms the hydroxy compound 3c. Reaction of 3c with POCl₃ forms the corresponding chloro derivative 3d. Suzuki-Miyaura coupling of 3d with boronate ester 2i provides compound 3e. Buchwald-Hartwig amination of 3e with aniline 2g provides adduct 3f. Desilylation of 3f with tetrabutylammonium fluoride yields alcohol 3g. Reaction of 3g with methanesulfonyl chloride in the presence of triethylamine generates the corresponding mesylate 3h. Finally, reaction of 3h with W—H produces compounds of the Formula III.

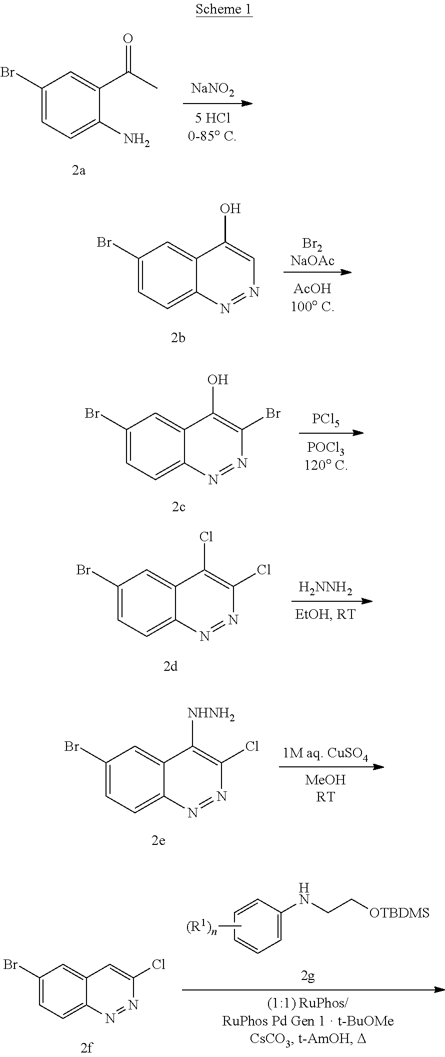

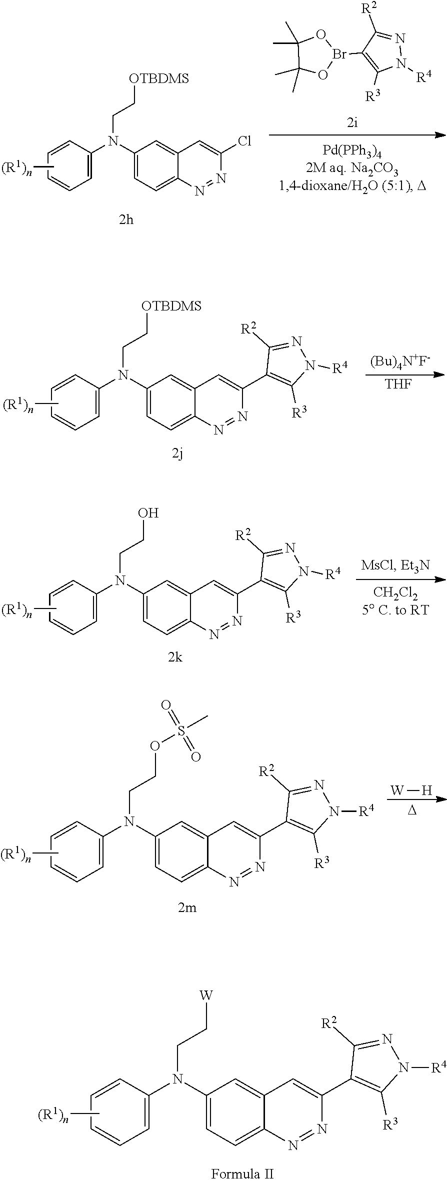

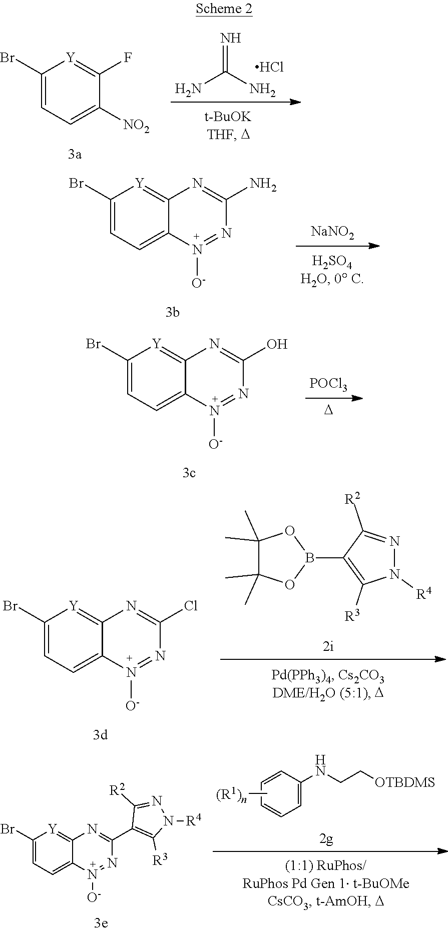

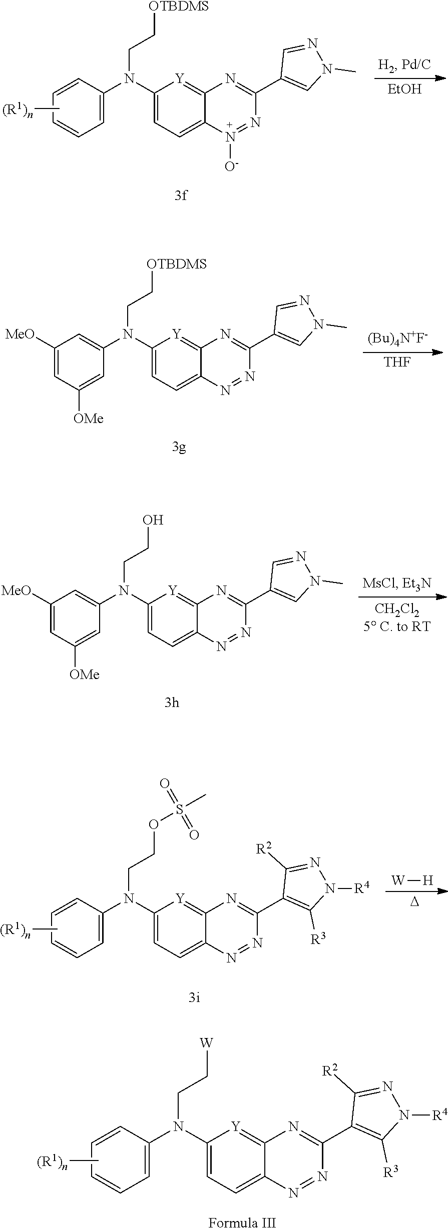

### EXAMPLES

The following non-limiting Examples further illustrate certain aspects of the present invention. These compounds are prepared according to the general synthetic schemes described above.

### Example 1

### N¹-(3,5-Dimethoxyphenyl)-N²-isopropyl-N¹-(3-(1-methyl-1H-pyrazol-4-yl)cinnolin-6-yl)ethane-1,2-diamine (1)

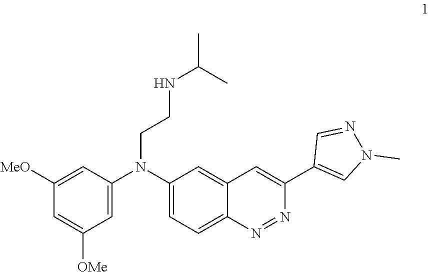 [SMILES: COc1cc(OC)cc(N(CCNC(C)C)c2ccc3nnc(-c4cnn(C)c4)cc3c2)c1]

Example 1 was prepared as shown below in Scheme 3.

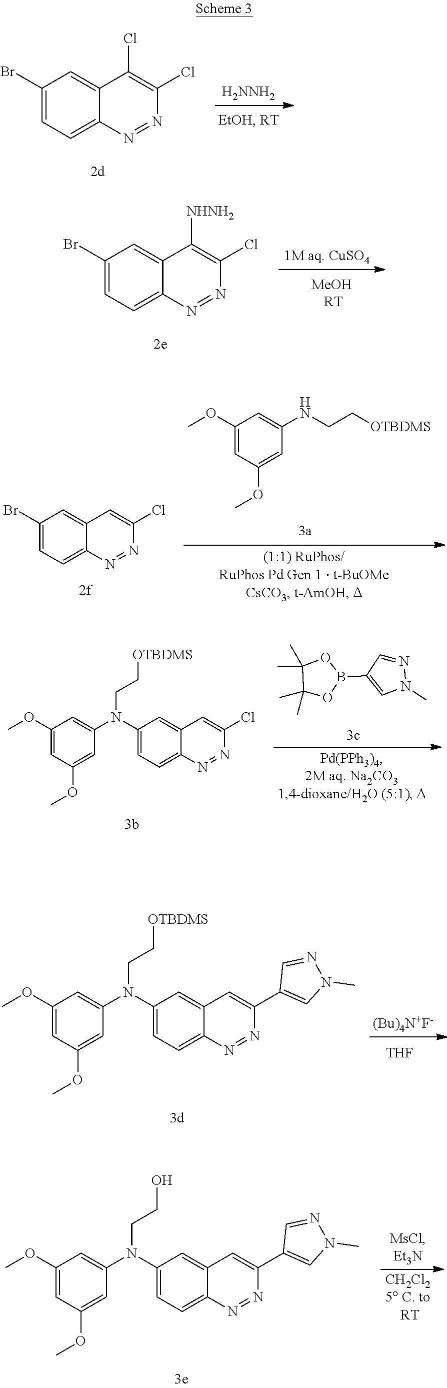

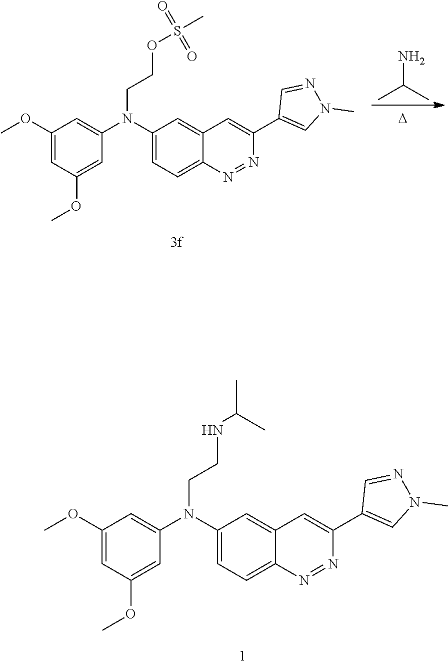

6-Bromo-3-chloro-4-hydrazinylcinnoline (2e). A slurry of 6-bromo-3,4-dichlorocinnoline (2d; CAS #2065250-59-9; 232 mg, 0.835 mmol) in anhyd. ethanol (4.0 mL) was treated with anhyd. hydrazine (262 μL, 8.35 mmol) and stirred under nitrogen at room temperature. After 1.5 h, the reaction mixture was concentrated in vacuo and the residue was partitioned between a mixture of CH₂Cl₂/MeOH (7:3) and water. The layers were separated and the aqueous layer was extracted again with CH₂Cl₂/MeOH (7:3). The combined organic extracts were washed with water, dried (Na₂SO₄), filtered and concentrated in vacuo to afford 168 mg (74%) of 6-bromo-3-chloro-4-hydrazinylcinnoline (2e) as a tan solid: MS (m/z) MH+=273; 1H NMR (300 MHz, DMSO-d₆): δ 11.17 (s, 1H), 8.34 (d, J=2.1 Hz, 1H), 7.50 (dd, J=8.8, 2.1 Hz, 1H), 6.94 (d, J=8.8 Hz, 1H), 6.87 (s, 2H).

6-Bromo-3-chlorocinnoline (2f). A stirring slurry of 6-bromo-3-chloro-4-hydrazinylcinnoline (2e; 195 mg, 0.384 mmol) in methanol (7.7 mL) was treated at room temperature with 1 M aq. CuSO₄·5H₂O (0.384 mL, 0.384 mmol). After 1 h, the resulting solution was concentrated in vacuo and the residue was partitioned between CH₂Cl₂ (20 mL) and 3% aq. NH₄OH (15 mL). The basic aq. layer was extracted twice with CH₂Cl₂ (10 mL) and the combined CH₂Cl₂ extracts were extracted twice with 3% aq. NH₄OH (15 mL), brine (20 mL), dried (Na₂SO₄), filtered and concentrated in vacuo. The residue was and chromatographed on silica gel eluting with 100% CH₂Cl₂ to provide 54 mg (58%) of 6-bromo-3-chlorocinnoline (2f) as a pink shiny solid: MS (m/z) MH+=243; ¹H NMR (300 MHz, CDCl₃): δ 8.41 (ddd, J=9.1, 0.9, 0.6 Hz, 1H), 7.98 (d, J=2.0 Hz, 1H), 7.93 (dd, J=9.1, 2.0 Hz, 1H), 7.85 (d, J=0.7 Hz, 1H).

N-(2-((tert-Butyldimethylsilyl)oxy)ethyl)-3-chloro-N-(3,5-dimethoxyphenyl)cinnolin-6-amine (3b). N-(2-((tert-Butyldimethylsilyl)oxy)ethyl)-3,5-dimethoxyaniline (3a; CAS #1346245-09-7; 43 mg, 0.131 mmol), 6-bromo-3-chlorocinnoline (2f; 32 mg, 0.131 mmol), CsCO₃ (40 mg, 0.394 mmol) and anhyd. tert-amyl alcohol (1.31 mL) were added to a microwave reaction tube and degassed by sparging with nitrogen thorough the reaction mixture for 1 h. RuPhos (6.1 mg; 0.0131 mmol) and RuPhos Pd Gen 1·t-BuOMe (10.7 mg, 0.0131 mmol) were added and the reaction mixture was heated at 85° C. in a microwave reactor for 3 h. The reaction mixture was cooled to room temperature, concentrated in vacuo and partitioned between CH₂Cl₂ and water. The water layer was extract twice with CH₂Cl₂ and the combined organic extracts were dried (Na₂SO₄), filtered and concentrated in vacuo. The residue was chromatographed on silica gel eluting with a gradient of 0-5% MeOH in CH₂Cl₂ to provide 50 mg (80%) of N-(2-((tert-butyldimethylsilyl)oxy)ethyl)-3-chloro-N-(3,5-dimethoxyphenyl)cinnolin-6-amine (3b) as viscous amber oil: MS (m/z) MH+=474; ¹H NMR (300 MHz, CDCl₃): δ 8.13 (d, J=9.6 Hz, 1H), 7.54 (d, J=0.7 Hz, 1H), 7.39 (dd, J=9.6, 2.6 Hz, 1H), 6.79 (d, J=2.6 Hz, 1H), 6.41 (s, 3H), 3.98 (t, J=5.5 Hz, 2H), 3.87 (t, J=5.5 Hz, 2H), 3.79 (s, 6H), 0.85 (s, 9H), 0.02 (s, 6H).

N-(2-((tert-Butyldimethylsilyl)oxy)ethyl)-N-(3,5-dimethoxyphenyl)-3-(1-methyl-1H-pyrazol-4-yl)cinnolin-6-amine (3d). N-(2-((tert-Butyldimethylsilyl)oxy)ethyl)-3-chloro-N-(3,5-dimethoxyphenyl)cinnolin-6-amine (3b; 43 mg, 0.907 mmol), 1-methyl-4-(4,4,5,5-tetramethyl-1,3,2-dioxaborolan-2-yl)-1H-pyrazole (3c; 57 mg, 0.272 mmol), 2 M aq. Na₂CO₃ (272 μL, 0.544 mmol) and 1,4-dioxane (1.09 mL) were combined in a microwave reaction tube and degassed by sparging with nitrogen thorough the reaction mixture for 1 h. Pd(PPh₃)₄ (31 mg, 0.0272 mmol) was added and the reaction mixture was heated in a microwave reactor at 150° C. After 30 min., the reaction mixture was cooled to room temperature, concentrated in vacuo and partitioned between CH₂Cl₂ and water. The aqueous layer was extract twice with CH₂Cl₂ and the combined organic extracts were dried (Na₂SO₄), filtered and concentrated in vacuo. The residue was chromatographed on silica gel eluting with a gradient of 0-5% MeOH in CH₂Cl₂ to provide 22 mg (57%) of N-(2-((tert-butyldimethylsilyl)oxy)ethyl)-N-(3,5-dimethoxyphenyl)-3-(1-methyl-1H-pyrazol-4-yl)cinnolin-6-amine (3d) as a thick brown oil: MS (m/z) MH+=520; ¹H NMR (300 MHz, CDCl₃): δ 8.21 (s, 1H), 8.13 (d, J=9.5 Hz, 1H), 8.05 (d, J=0.6 Hz, 1H), 7.61 (d, J=0.6 Hz, 1H), 7.35 (dd, J=9.5, 2.6 Hz, 1H), 6.91 (d, J=2.6 Hz, 1H), 6.43 (d, J=2.2 Hz, 2H), 6.38 (dd, J=4.4, 2.2 Hz, 1H), 4.01 (s, 3H), 4.00 (t, J=5.5 Hz, 2H), 3.88 (t, J=5.5 Hz, 2H), 3.79 (s, 6H), 0.87 (s, 9H), 0.24 (s, 6H).

2-((3,5-Dimethoxyphenyl)(3-(1-methyl-1H-pyrazol-4-yl)cinnolin-6-yl)amino)ethan-1-ol (3e). A solution of N-(2-((tert-butyldimethylsilyl)oxy)ethyl)-N-(3,5-dimethoxyphenyl)-3-(1-methyl-1H-pyrazol-4-yl)cinnolin-6-amine (3d; 25 mg, 0.0482 mmol) was treated at room temperature with 1 M tetrabutylammonium fluoride in THE (59 μL, 0.059 mmol) and stirred at room temperature for 1.5 h. The reaction mixture was concentrated in vacuo and partitioned between ethyl acetate (7 mL) and water (7 mL). The aqueous layer was extract twice with ethyl acetate (7 mL) and the combined organic extracts were dried (Na₂SO₄), filtered and concentrated in vacuo to give 2-((3,5-dimethoxyphenyl)(3-(1-methyl-1H-pyrazol-4-yl)cinnolin-6-yl)amino)ethan-1-ol (3e) as an amber glass: MS (m/z) MH+=406; ¹H NMR (300 MHz, CDCl₃): δ 8.17 (s, 1H), 7.97 (d, J=9.5 Hz, 1H), 7.88 (s, 1H), 7.46 (s, 1H), 7.16 (dd, J=9.5, 2.5 Hz, 1H), 6.88 (d, J=2.5 Hz, 1H), 6.45 (d, J=2.2 Hz, 2H), 6.38 (dd, J=4.4, 2.2 Hz, 1H), 4.08-4.00 (overlapping m, 4H), 3.98 (s, 3H), 3.77 (s, 7H).

2-((3,5-Dimethoxyphenyl)(3-(1-methyl-1H-pyrazol-4-yl)cinnolin-6-yl)amino)ethyl methanesulfonate (3f). A solution of 2-((3,5-dimethoxyphenyl)(3-(1-methyl-1H-pyrazol-4-yl)cinnolin-6-yl)amino)ethan-1-ol (3e; 20 mg, 0.0493 mmol), triethylamine (21 μL, 0.148 mmol) in CH₂Cl₂ (1.0 mL) was cooled to 5 C and treated with methanesulfonyl chloride (76 μL, 0.099 mmol) and stirred at 5° C. for 1 h. The reaction mixture was then warmed to room temperature and stirred for 1 h and then quenched with water (10 mL). The layers were separated and the aqueous layer was extracted twice with CH₂Cl₂ (5 mL). The combine organic extracts were washed with water (10 mL), dried (Na₂SO₄), filtered and concentrated in vacuo to afford 2-((3,5-dimethoxyphenyl)(3-(1-methyl-1H-pyrazol-4-yl)cinnolin-6-yl)amino)ethyl methanesulfonate (3f) as a thick red oil: MS (m/z) MI-1±=484.

N¹-(3,5-Dimethoxyphenyl)-N²-isopropyl-N¹-(3-(1-methyl-1H-pyrazol-4-yl)cinnolin-6-yl)ethane-1,2-diamine (1). 2-((3,5-Dimethoxyphenyl)(3-(1-methyl-1H-pyrazol-4-yl)cinnolin-6-yl)amino)ethyl methanesulfonate (3f; 24 mg, 0.0493 mmol) was combined with isopropylamine (5 mL, 61 mmol) in a sealed pressure tube and heated while stirring at 90° C. for 23 h. The reaction mixture was cooled to room temperature and concentrated in vacuo. The residue was chromatographed on silica gel eluting with a gradient of 0-10% MeOH in CH₂Cl₂ to furnish 14 mg (64%) of N¹-(3,5-dimethoxyphenyl)-N²-isopropyl-N¹-(3-(1-methyl-1H-pyrazol-4-yl)cinnolin-6-yl)ethane-1,2-diamine (1) as an amber glass: MS (m/z) MH⁺=447; ¹H NMR (300 MHz, CDCl₃): δ 8.21 (s, 1H), 8.08 (d, J=9.5 Hz, 1H), 8.05 (s, 1H), 7.66 (s, 1H), 7.22 (dd, J=9.5, 2.5 Hz, 1H), 6.95 (d, J=2.5 Hz, 1H), 6.38 (s, 3H), 4.10 (t, J=7.0 Hz, 2H), 4.04 (s, 3H), 3.77 (s, 6H), 3.13-2.91 (overlapping m, 3H), 1.19 (d, J=6.3 Hz, 6H), 0.87 (broad s, 1H).

### In Vitro Assays

Biochemical Assay. The RBC HotSpot Kinase Assays were performed by Reaction Biology Corporation, 1 Great Valley Parkway, Suite 2 Malvern, PA 19355, USA. The reaction buffer was 20 mM HEPES (pH 7.5), 10 mM MgCl₂, 1 mM EGTA, 0.02% Brij35, 0.02 mg/mL BSA, 0.1 mM Na₃VO₄, 2 mM DTT, 1% DMSO. The required cofactors are added individually to each kinase reaction. The enzyme concentration was 1.75 nM and the ATP Kₘ was 5 μM. The substrate was Poly (Glu, Tyr) sodium salt and the concentration was 0.2 mg/mL (Sigma; cat. #P7244) where the Glu:Tyr (4:1) molecular weight was 5,000-20,000. The test compounds were dissolved in 100% DMSO and tested in 10-dose IC₅₀ mode with a 3-fold serial dilution starting at 1 μM. The reference standard, staurosporine, was tested in 10-dose IC₅₀ mode with 4-fold serial dilution starting at 20 μM. Reactions were carried out at 10 μM ATP. The serial dilution was conducted by Integra Viaflo Assist in DMSO. The reaction procedure was conducted as follows:
1) Prepare substrate in freshly prepared Reaction Buffer.
2) MnCl₂ (2 mM) was added as a co-factor listed as above.
3) Deliver kinase into the substrate solution and gently mix.
4) Deliver compounds in 100% DMSO into the kinase reaction mixture by Acoustic technology (Echo550; nanoliter range), incubate for 20 min at RT.
5) Deliver 33P-ATP (specific activity 10 μCi/μL) into the reaction mixture to initiate the reaction.
6) Incubate for 2 hours at RT.
7) Detect radioactivity by the filter-binding method.
8) Kinase activity data was expressed as the percent remaining kinase activity in the test sample compared to vehicle (DMSO) reactions. Curve fits were performed where the enzyme activities at the highest concentration of compounds were less than 65%. IC₅₀ values and curve fits were obtained using Prism (GraphPad Software) and the results are shown below:

| | FGFR1 | FGFR2 | FGFR3 | FGFR4 |
|---|---|---|---|---|
| Compound | IC₅₀ (nM) | IC₅₀ (nM) | IC₅₀ (nM) | IC₅₀ (nM) |
| staurosporine | 2.1 | 0.80 | 8.9 | 41 |
| (reference standard) | | | | |
| Example 1 | 7.3 | 4.3 | 7.6 | 11 |

Cellular Assay. The RBC NanoBRET Assay for Target Engagement was performed by Reaction Biology Corporation, 1 Great Valley Parkway, Suite 2 Malvern, PA 19355, USA. The assay was conducted in HEK293 cells transiently transfected with FGFR1-NanoLuc Fusion vector. The HEK293 cells (ATCC) were transfected with 1 μg of FGFR1-NanoLuc Fusion vector in 384-well format and the transfected cells were subsequently treated with the test compounds (starting at 10 μM, 10-dose with 3-fold dilution) for 1 hour using CTx-0294885 as the reference standard and K5 (1 μM) was used as the tracer. FGFR1 target engagement was measured and the curve fits were performed only when % NanoBret signal at the highest concentration of compounds was less than 55%. The results are shown below:

| Compound | FGFR1 IC₅₀ (nM) |
|---|---|
| CTx-0294885 | 35 |
| (reference standard) | |
| Example 1 | 9.5 |

### In Vivo Assay

Pharmacokinetics Assay. A pharmacokinetics assay of Example 1 was performed by WuXi AppTec Inc., 6 Cedarbrook Drive, Cranbury, NJ 08512 USA. This study was conducted in 6 to 8-week-old male CD-1 mice (Hilltop Laboratories). Animals were group housed during acclimation and throughout the study. The animal room environment was controlled according to facility operation (temperature: 20 to 26° C.; relative humidity: 30 to 70%; lighting: 12 hour light/dark cycle). Temperature, relative humidity, and lighting were monitored by PointView Environmental Monitoring System. Animals were fed certified pellet diet (Certified Rodent Diet #5002, LabDiet). The diet lot number and specifications were recorded in study notebook and archived at WuXi AppTec. Water (reverse osmosis) was provided to the animals ad libitum. The animals were fasted overnight and food was returned 4 hours post-dose.

An appropriate amount of Example compound 1 above (N¹-(3,5-Dimethoxyphenyl)-N²-isopropyl-N¹-(3-(1-methyl-1H-pyrazol-4-yl)cinnolin-6-yl)ethane-1,2-diamine) was accurately weighed and mixed with an appropriate volume of 20% hydroxypropyl-(3-cyclodextrin; 80% H₂O (w/v) to give either a clear solution or uniform suspension (PO only). Blood samples of about 30-40 μL were collected from peripheral veins at pre-defined time points. into pre-chilled micro-tubes containing K₂EDTA as anti-coagulant and kept on ice until centrifugation. The blood samples were centrifuged at 4° C., 3000g for 15 min within half an hour of collection. Plasma was collected into 96-well plate(s), quickly frozen on dry ice and stored at −70±10° C. until LC-MS/MS analysis. The LC-MS/MS analytical method is described as follows:

| Instrument | Triple Quad 6500+ | |
|---|---|---|
| Matrix | Male CD-1 mouse plasma (EDTA-K2) | |
| Internal | 100 ng/mL Labetalol & 100 ng/mL Tolbutamide & | |
| standard(s) | 100 ng/mL Diclofenac in ACN | |
| MS conditions | ESI: positive | |
| | SRM detection | |
| | Example 1 | [M + H] + m/z 447.3 > 362.4 |
| | Labelatol (IS) | [M + H] + m/z 329.2 > 162.1 |
| UPLC | Mobile Phase: | |
| conditions | Mobile Phase A: 0.1% FA in Water | |
| | Mobile Phase B: 0.1% FA in ACN | |
| | Time (min) | Mobile Phase B (%) |
| | 1.10 | 98 |
| | 1.50 | 98 |
| | 1.51 | 10 |
| | 2.00 | Stop |
| | Column: Waters ACQUITY UPLC BEH C18 2.1*50 | |
| | mm, 1.7 μm | |
| | Flow rate: 0.6000 mL/min | |
| | Retention time: | |
| | Example 1 | 0.95 min |
| | Labelatol (IS) | 0.98 min |

| | |
|---|---|
| Sample | 1). An aliquot of 20 μL sample was protein |
| preparation | precipitated with 200 μL IS solution, the mixture was |
| | vortex-mixed well and centrifuged at 4000 rpm for 15 |
| | min, 4° C. An aliquot of 100 μL supernatant was |
| | transferred to sample plate and mixed with 100 μL |
| | water, then the plate was shaken at 800 rpm for 10 |
| | min. |
| | 2). 0.1-0.3 μL supernatant was then injected for |
| | LC-MS/MS analysis. |
| Calibration | 1.00-3000 ng/mL for BPI-209-AA-2 in male CD-1 |
| curve | mouse plasma (EDTA-K2) |
| | 1.00-3000 ng/mL for BPI-224-AA-3 in male CD-1 |
| | mouse plasma (EDTA-K2) |

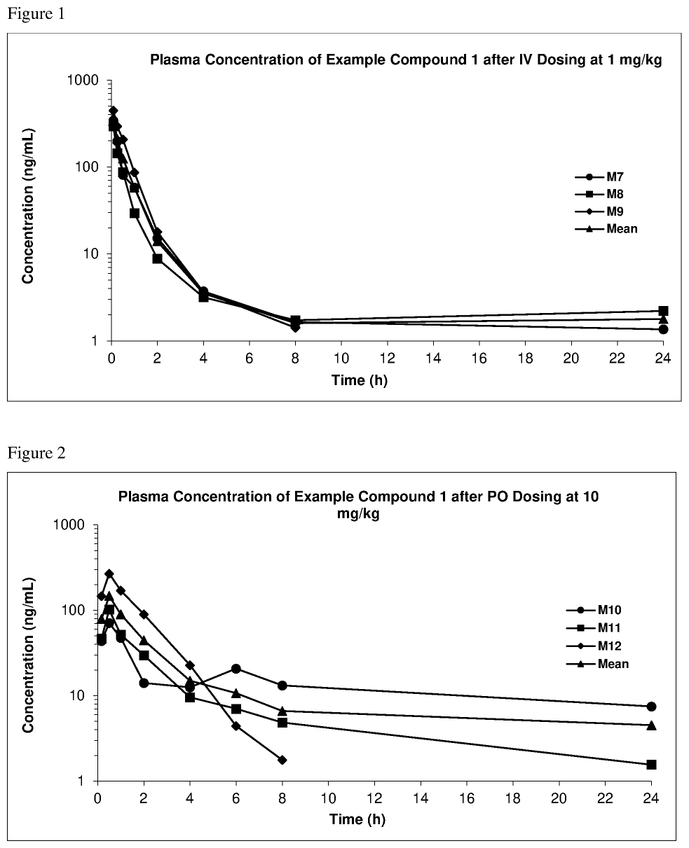

The pharmacokinetics of Example Compound 1 were analyzed using the Phoenix WinNonlin software (version 6.3) based on the non-compartmental analysis model and the results are shown below in FIGS. 1 and 2 and in Tables 1 and 2. M7, M8, M9, etc. refer to particular mouse subjects used in the study.

TABLE 1
Pharmacokinetics of Example 1 in the Mouse (ng/mL)
IV (1.00 mg/kg)

| | M7 | M8 | M9 | Mean | | SD | CV (%) |
|---|---|---|---|---|---|---|---|
| Time (h) | | | | | | | |
| 0.0830 | 341 | 294 | 444 | 360 | ± | 76.7 | 21.3 |
| 0.250 | 197 | 143 | 292 | 211 | ± | 75.4 | 35.8 |
| 0.500 | 80.8 | 86.8 | 206 | 125 | ± | 70.6 | 56.7 |
| 1.00 | 58.0 | 29.3 | 86.4 | 57.9 | ± | 28.6 | 49.3 |
| 2.00 | 15.2 | 8.83 | 17.9 | 14.0 | ± | 4.66 | 33.3 |
| 4.00 | 3.72 | 3.17 | 3.64 | 3.51 | ± | 0.297 | 8.47 |
| 8.00 | 1.65 | 1.73 | 1.42 | 1.60 | ± | 0.161 | 10.1 |
| 24.0 | 1.36 | 2.21 | BQL | 1.79 | ± | ND | ND |
| PK Parameters | | | | | | | |
| Rsq_adj | 0.836 | 0.781 | 0.841 | 0.819 | ± | 0.0333 | 4.06 |
| No. points used for T_{1/2} | 5.00 | 3.00 | 7.00 | 5.00 | ± | 2.00 | 40.0 |
| C₀ (ng/mL) | 448 | 421 | 547 | 472 | ± | 66.4 | 14.1 |
| T_{1/2} (h) | 1.34 | 2.72 | 0.948 | 1.67 | ± | 0.934 | 55.9 |
| Vdₛₛ (L/kg) | 12.2 | 23.3 | 2.84 | 12.8 | ± | 10.2 | 80.0 |
| Cl (mL/min/kg) | 73.0 | 84.8 | 54.7 | 70.8 | ± | 15.2 | 21.4 |
| Tₗₐₛₜ (h) | 24.0 | 24.0 | 8.00 | 18.7 | ± | 9.24 | 49.5 |
| AUC₀₋ₗₐₛₜ (ng · h/mL) | 226 | 188 | 303 | 239 | ± | 58.6 | 24.5 |
| AUC_{0-inf} (ng · h/mL) | 228 | 197 | 305 | 243 | ± | 55.6 | 22.9 |
| MRT₀₋ₗₐₛₜ (h) | 2.53 | 3.50 | 0.812 | 2.28 | ± | 1.36 | 59.7 |
| MRT_{0-inf} (h) | 2.80 | 4.58 | 0.866 | 2.75 | ± | 1.86 | 67.6 |
| AUC_{Extra} (%) | 1.15 | 4.42 | 0.637 | 2.07 | ± | 2.05 | 99.2 |
| AUMC_{Extra} (%) | 10.6 | 26.9 | 6.89 | 14.8 | ± | 10.7 | 71.9 |

TABLE 2
Pharmacokinetics of Example 1 in the Mouse (ng/mL)
PO (10.0 mg/kg)

| | M10 | M11 | M12 | Mean | | SD | CV (%) |
|---|---|---|---|---|---|---|---|
| Time (h) | | | | | | | |
| 0.167 | 43.6 | 46.8 | 147 | 79.1 | ± | 58.8 | 74.3 |
| 0.500 | 71.0 | 102 | 269 | 147 | ± | 107 | 72.3 |
| 1.00 | 47.6 | 51.9 | 170 | 89.8 | ± | 69.5 | 77.3 |
| 2.00 | 14.1 | 29.7 | 89.5 | 44.4 | ± | 39.8 | 89.6 |
| 4.00 | 12.5 | 9.57 | 22.8 | 15.0 | ± | 6.95 | 46.5 |
| 6.00 | 20.7 | 7.02 | 4.44 | 10.7 | ± | 8.74 | 81.5 |
| 8.00 | 13.2 | 4.84 | 1.77 | 6.60 | ± | 5.92 | 89.6 |
| 24.0 | 7.49 | 1.56 | BQL | 4.53 | ± | ND | ND |
| PK Parameters | | | | | | | |
| Rsq_adj | 0.757 | 0.962 | 0.991 | 0.904 | ± | 0.128 | 14.1 |
| No. points used for T_{1/2} | 3.00 | 3.00 | 5.00 | 3.67 | ± | 1.15 | 31.5 |
| Cₘₐₓ (ng/mL) | 71.0 | 102 | 269 | 147 | ± | 107 | 72.3 |
| Tₘₐₓ (h) | 0.500 | 0.500 | 0.500 | 0.500 | ± | 0 | 0 |
| T_{1/2} (h) | 14.3 | 8.81 | 1.03 | 8.05 | ± | 6.68 | 82.9 |
| Tₗₐₛₜ (h) | 24.0 | 24.0 | 8.00 | 18.7 | ± | 9.24 | 49.5 |
| AUC₀₋ₗₐₛₜ (ng · h/mL) | 333 | 216 | 441 | 330 | ± | 113 | 34.1 |
| AUC_{0-inf} (ng · h/mL) | 488 | 235 | 443 | 389 | ± | 135 | 34.6 |
| MRT₀₋ₗₐₛₜ (h) | 9.01 | 4.78 | 1.60 | 5.13 | ± | 3.72 | 72.4 |
| MRT_{0-inf} (h) | 20.3 | 7.47 | 1.65 | 9.81 | ± | 9.55 | 97.3 |
| AUC_{Extra} (%) | 31.7 | 8.42 | 0.593 | 13.6 | ± | 16.2 | 119 |
| AUMC_{Extra} (%) | 69.7 | 41.4 | 3.41 | 38.2 | ± | 33.3 | 87.2 |
| Bioavailability (%)^{a} | | | | 13.8 | | | |

ND = Not determined due to inadequately defined terminal elimination phase or insufficient number of values
BQL = Below the lower limit of quantitation (LLOQ)
If the adjusted rsq (linear regression coefficient of the concentration value on the terminal phase) is less than 0.9, T½ might not be accurately estimated.
If the % AUC_{Extra} > 20%, AUC_{0-inf}, Cl, MRT_{0-inf} and Vdₛₛ might not be accurately estimated.
If the % AUMC_{Extra} > 20%, MRT_{0-inf} and Vdₛₛ might not be accurately estimated.
^{a}Bioavailability (%) was calculated using AUC_{0-inf} (% AUC_{Extra} < 20%) or AUC₀₋ₗₐₛₜ (% AUC_{Extra} > 20%) with nominal dose (80% < dose accuracy < 120%) or administered dose (80% > dose accuracy> 120%)

1. A compound of the Formula I: 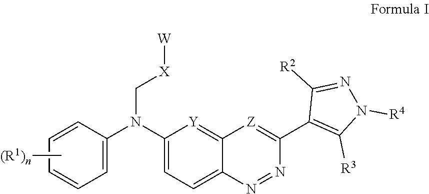
or a pharmaceutically acceptable salt, solvate, or prodrug thereof, wherein:
W is selected from H, OH, NH₂, NH(C₁₋₆alkyl), 1-azetindinyl, 1-pyrrolidinyl, 1-piperidinyl, 2-pyridyl, 1,2,3,6-tetrahydro-1-pyridinyl, 2-pyrimidinyl, 4-pyrimidinyl, 2-thiazolo, and phenyl optionally substituted with one or more of OH, halogen, CF₃, C₁₋₄alkyl, O(C₁₋₄alkyl), (C₁₋₄alkyl)OH, CN, CH₂CN, C(O)NH₂, C(O)NH(C₁₋₄alkyl), C(O)N(C₁₋₄alkyl)₂, NH₂, NH(C₁₋₄alkyl), N(C₁₋₄alkyl)₂, NHSO₂(C₁₋₄alkyl), NHSO₂NH(C₁₋₄alkyl) and NHSO₂N(C₁₋₄alkyl)₂;
X is selected from a bond, methylene, ethylene, and ethynylene;
Y and Z are the same or different and are selected from CH and N;
each R¹ is independently selected from is independently selected from H, OH, halogen, CN, C₁₋₄alkyl, O(C₁₋₄alkyl), NH(C₁₋₄alkyl), and N(C₁₋₄alkyl)₂;
n is an integer selected from 1-5;
R² and R³ are the same or different and are selected from H and C₁₋₄alkyl; and
R⁴ is selected from H, C₁₋₄alkyl, C₃₋₆cycloalkyl, C₁₋₄alkylOH, CH₂C(O)NHC₁₋₂alkyl, CH₂C(O)N(C₁₋₂alkyl)₂, CH₂(SO)₂C₁₋₂alkyl, 4-(λ³-methyl)tetrahydro-2H-pyran, 4-(λ³-methyl)piperidine, CH₂P(O)(C₁₋₂alkyl)₂, CH₂P(O)(OC₁₋₂alkyl)₂, CH₂P(O)(OC₁₋₂alkyl)OH and CH₂P(O)(OH)₂.

2. A compound of Formula II: 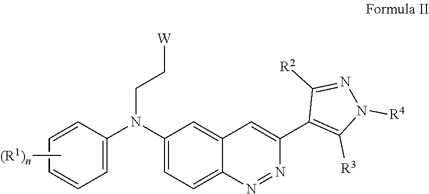
or a pharmaceutically acceptable salt, solvate, or prodrug thereof, wherein:
W is selected from H, OH, NH₂, NH(C₁₋₆alkyl), 1-azetindinyl, 1-pyrrolidinyl, 1-piperidinyl, 2-pyridyl, 1,2,3,6-tetrahydro-1-pyridinyl, 2-pyrimidinyl, 4-pyrimidinyl, 2-thiazolo, and phenyl optionally substituted with one or more of OH, halogen, CF₃, C₁₋₄alkyl, O(C₁₋₄alkyl), (C₁₋₄alkyl)OH, CN, CH₂CN, C(O)NH₂, C(O)NH(C₁₋₄alkyl), C(O)N(C₁₋₄alkyl)₂, NH₂, NH(C₁₋₄alkyl), N(C₁₋₄alkyl)₂, NHSO₂(C₁₋₄alkyl), NHSO₂NH(C₁₋₄alkyl) and NHSO₂N(C₁₋₄alkyl)₂;
each R¹ is independently selected from is independently selected from H, OH, halogen, CN, C₁₋₄alkyl, O(C₁₋₄alkyl), NH(C₁₋₄alkyl), and N(C₁₋₄alkyl)₂;
n is an integer selected from 1-5;
R² and R³ are the same or different and are selected from H and C₁₋₄alkyl; and
R⁴ is selected from H, C₁₋₄alkyl, C₃₋₆cycloalkyl, C₁₋₄alkylOH, CH₂C(O)NHC₁₋₂alkyl, CH₂C(O)N(C₁₋₂alkyl)₂, CH₂(SO)₂C₁₋₂alkyl, 4-(λ³-methyl)tetrahydro-2H-pyran, 4-(λ³-methyl)piperidine, CH₂P(O)(C₁₋₂alkyl)₂, CH₂P(O)(OC₁₋₂alkyl)₂, CH₂P(O)(OC₁₋₂alkyl)OH and CH₂P(O)(OH)₂.

3. The compound of claim 2, selected from: 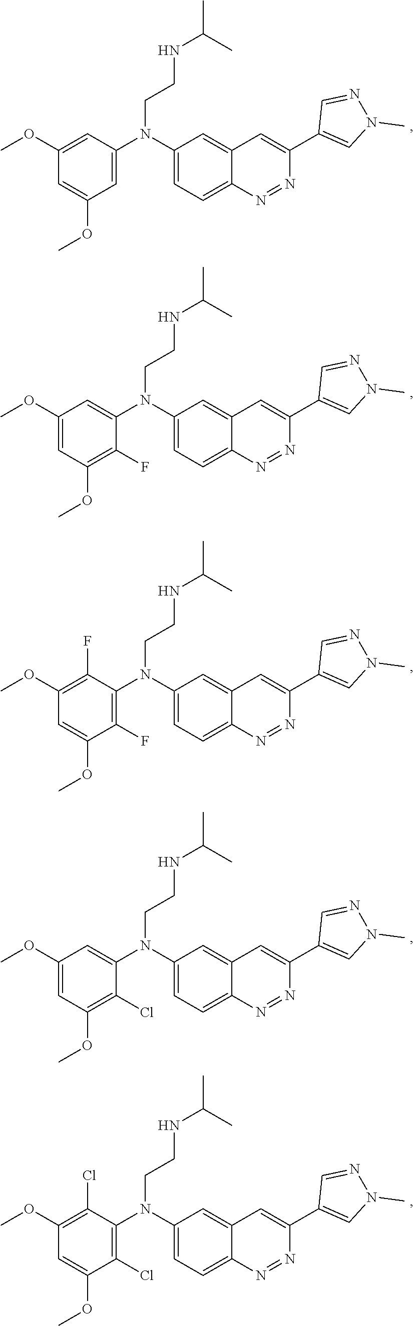 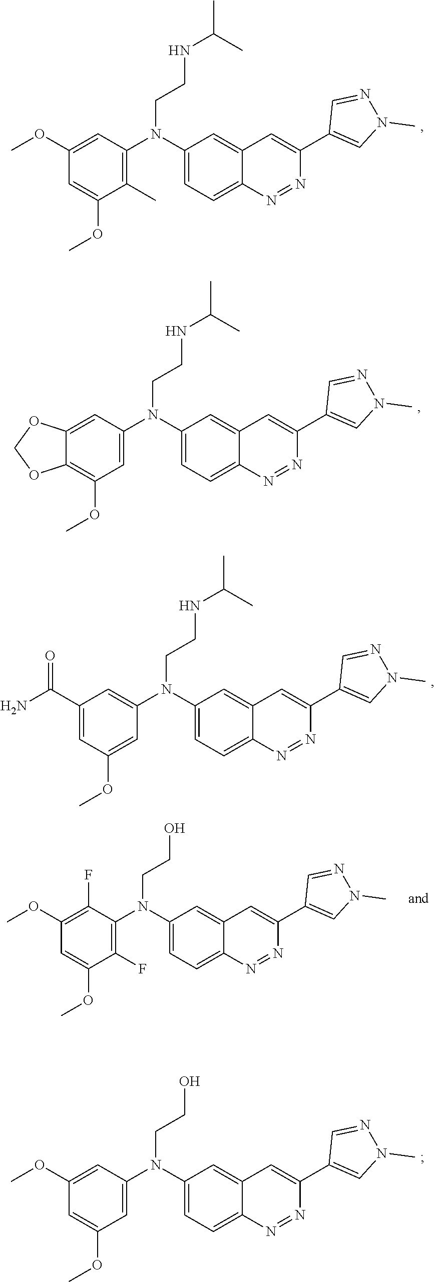
or a pharmaceutically acceptable salt, solvate, or prodrug thereof.

4. A compound of Formula III: 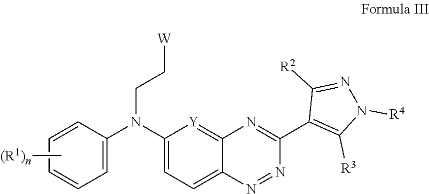
or a pharmaceutically acceptable salt, solvate, or prodrug thereof, wherein:
W is selected from H, OH, NH₂, NH(C₁₋₆alkyl), 1-azetindinyl, 1-pyrrolidinyl, 1-piperidinyl, 2-pyridyl, 1,2,3,6-tetrahydro-1-pyridinyl, 2-pyrimidinyl, 4-pyrimidinyl, 2-thiazolo, and phenyl optionally substituted with one or more of OH, halogen, CF₃, C₁₋₄alkyl, O(C₁₋₄alkyl), (C₁₋₄alkyl)OH, CN, CH₂CN, C(O)NH₂, C(O)NH(C₁₋₄alkyl), C(O)N(C₁₋₄alkyl)₂, NH₂, NH(C₁₋₄alkyl), N(C₁₋₄alkyl)₂, NHSO₂(C₁₋₄alkyl), NHSO₂NH(C₁₋₄alkyl) and NHSO₂N(C₁₋₄alkyl)₂;
X is selected from a bond, methylene, ethylene, and ethynylene;
Y is selected from CH and N;
each R¹ is independently selected from is independently selected from H, OH, halogen, CN, C₁₋₄alkyl, O(C₁₋₄alkyl), NH(C₁₋₄alkyl), and N(C₁₋₄alkyl)₂;
n is an integer selected from 1-5;
R² and R³ are the same or different and are selected from H and C₁₋₄alkyl; and
R⁴ is selected from H, C₁₋₄alkyl, C₃₋₆cycloalkyl, C₁₋₄alkylOH, CH₂C(O)NHC₁₋₂alkyl, CH₂C(O)N(C₁₋₂alkyl)₂, CH₂(SO)₂C₁₋₂alkyl, 4-(λ³-methyl)tetrahydro-2H-pyran, 4-(λ³-methyl)piperidine, CH₂P(O)(C₁₋₂alkyl)₂, CH₂P(O)(OC₁₋₂alkyl)₂, CH₂P(O)(OC₁₋₂alkyl)OH and CH₂P(O)(OH)₂.

5. The compound of claim 4, selected from: 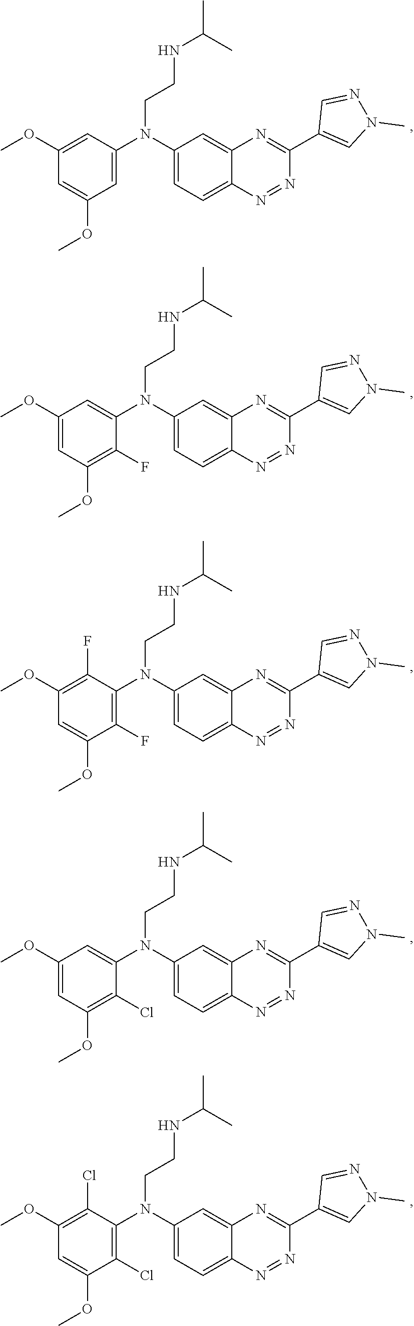 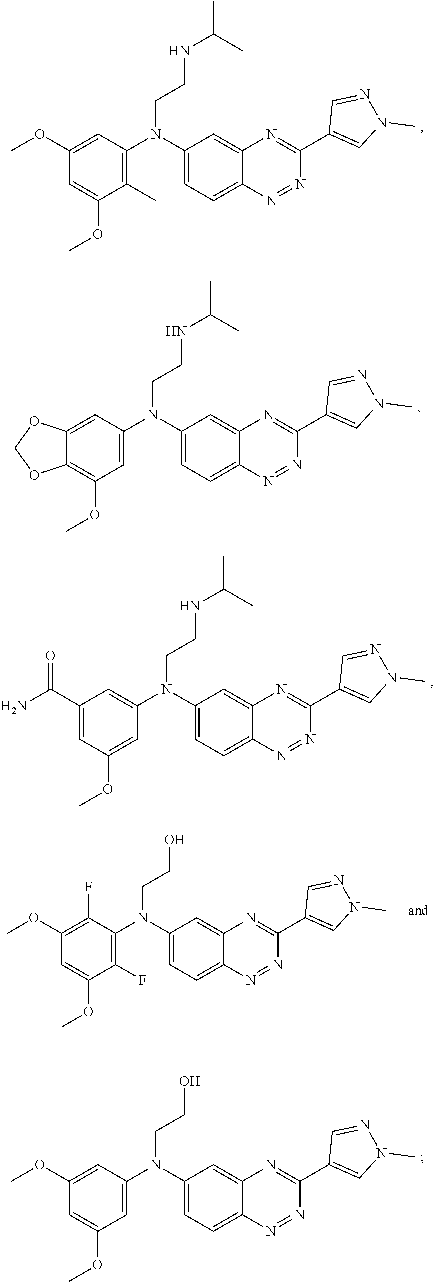
or a pharmaceutically acceptable salt, solvate, or prodrug thereof.

6. The compound of claim 4, selected from: 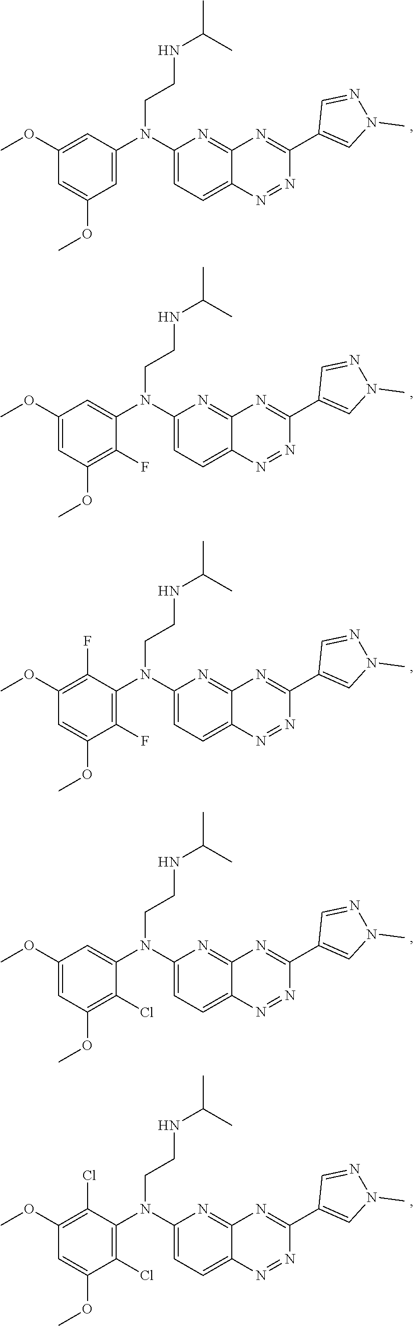 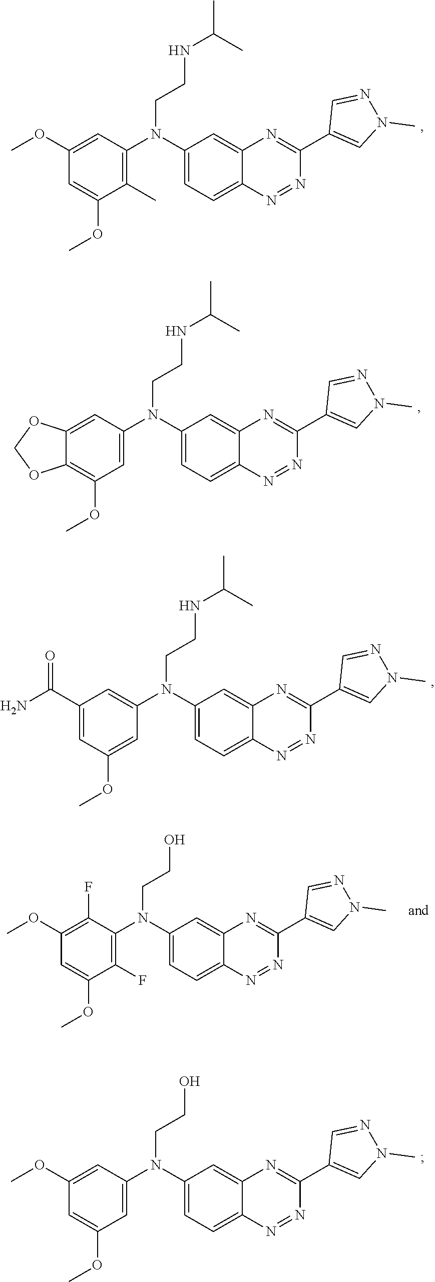
or a pharmaceutically acceptable salt, solvate, or prodrug thereof.

7. A pharmaceutical composition comprising a compound, salt, solvate, or prodrug of the compound of Formula I, together with a pharmaceutically acceptable carrier.

8. A method of treating a disease, disorder, or medical condition in a patient, comprising the step of providing to a patient in need thereof a therapeutic agent, wherein the therapeutic agent is a compound, salt, solvate, or prodrug thereof of the compound of Formula I.

9. The method of treating a disease, disorder, or medical condition in claim 8 in a patient, where diseases includes various cancers.

10. The method of treating a disease, disorder, or medical condition of claim 8 in a patient, where a disease, disorder, or medical condition is mediated through the fibroblast growth factor receptor (FGFR).

11. The method of treating a disease, disorder, or medical condition of claim 10 in a patient, where a disease, disorder, or medical condition is mediated through FGFR1-4.

12. The method of claim 9, wherein the cancer is selected from glioma (glioblastoma), acute myelogenous leukemia, acute myeloid leukemia, myelodysplastic/myeloproliferative neoplasms, sarcoma, chronic myelomonocytic leukemia, non-Hodgkin lymphoma, astrocytoma, melanoma, non-small cell lung cancer, cholangiocarcinomas, chondrosarcoma, colon cancer or pancreatic cancer.

13. The method of claim 8, further comprising administering to the patient in need thereof at least one additional therapeutic agent.
